(19) United States
(12) Patent Application Publication
(10) Pub. No.: US 2025/0058311 A1
(43) Pub. Date: Feb. 20, 2025

(54) CORE SHELL CATALYST AND METHOD FOR MANUFACTURING THE SAME

(71) Applicant: SABIC GLOBAL TECHNOLOGIES, B.V., Bergen op Zoom (NL)

(72) Inventors: Suman Kumar Sen, Bangalore (IN); Prashant Patil, Bangalore (IN); Surya Prakasa Rao Daliparthi, Bangalore (IN); Dwairath DHAR, Bangalore (IN); Amit Kumar Mondal, Hosur (IN); Paulus Johannes Maria Eijsbouts, Nieuwkuijk (NL); Rudolf Martinus Petrus van der Heijden, Geleen (NL); Gert-Jan Schoenmakers, Prinsenbeek (NL)

(21) Appl. No.: 18/721,053

(22) PCT Filed: Dec. 12, 2022
(86) PCT No.: PCT/EP2022/085305

§ 371 Date: Jun. 17, 2024

(30) Foreign Application Priority Data
Dec. 17, 2021 (EP) 21215714.3
Dec. 17, 2021 (EP) 21215716.8

(51) Int. Cl. B01J 37/02; B01J 21/08; B01J 35/40; B01J 35/53; C07C 67/42

(52) U.S. Cl. CPC B01J 37/0221; B01J 21/08; B01J 35/40; B01J 35/53; B01J 37/0219; C07C 67/42

(57) ABSTRACT

The present invention relates to a method for the manufacture of a core-shell catalyst comprising the steps of a. providing core particles, b. functionalizing at least part of the surface of the core particles with a functionalizing agent thereby forming functionalized core particles, c. graft polymerizing at least one of aromatic vinyl compounds onto the functionalized core particles thereby forming core-shell particles wherein the core is comprised of the core particles and the shell is comprised of graft polymerized aromatic vinyl compounds and d. activating the shell by using a sulfonating agent wherein the core particles comprise or consists of glass particles and wherein the core particles are hydroxylated prior to step b). The present invention further relates to the use of the core-shell catalyst for the manufacture of bisphenol A by reacting phenol with acetone for increasing the selectivity towards the formation of p,p-bisphenol A.

### CROSS REFERENCE TO RELATED APPLICATIONS

This application is a National Stage application of PCT/EP2022/085305, filed Dec. 12, 2022, which claims the benefit of European Application No. 21215714.3 and of European Application No. 21215716.8, both filed Dec. 17, 2021, both of which are incorporated by reference in their entirety herein.

### BACKGROUND

The present invention relates to a method for the manufacture of a core shell catalyst and a core shell catalyst obtainable by the said method. The present invention further relates to a method for the manufacture of a bisphenol compound and the use of a core-shell catalyst.

Industrial production of bisphenol A (BPA) typically involves a process whereby a mixture of excess phenol and acetone is passed through a fixed-bed reactor filled with divinyl benzene cross-linked sulfonated polystyrene ion exchange resin catalyst. The direction of flow of the mixture may be either downwards or upwards, also referred to as down-flow and up-flow respectively. Each feed direction has its own advantages and disadvantages.

The common technique in the industry for the manufacture of a bisphenol compound is to operate the fixed bed reactor in down flow mode where a mixture of phenol and ketone is passed through a fixed bed of ion-exchange resin particles. Such mode of operation is limited in that the weight hourly space velocity (WSHV) cannot be increased far above 1.0 because catalyst particles might compress under a higher forced throughput resulting in an exponentially increasing pressure drop. Moreover, compression of the catalyst bed under pressure can promote the formation of flow channels so that flow through the reactor is not uniform. As a result, the quantity of catalyst used may not be fully utilized and acetone conversion rate may drop. This problem has been addressed in WO 2000/050372 and U.S. Pat. No. 5,395,857A both disclosing a catalyst bed comprised of two layers.

In order to solve this problem a reactor may be operated in an up flow manner but because of the size and the relatively low density of commonly used ion exchange resin catalyst particles, an increase in throughput results in channeling or even fluidization of the fixed bed, which in turn results in a drop in acetone conversion.

This problem is addressed in WO 2020/099285 which discloses a catalyst for the manufacture of a bisphenol from phenol and a ketone, comprising particles having a core and a shell, wherein the shell comprises an ion exchange resin covering the core at least in part and wherein the core has a density that is higher than the density of the ion exchange resin, wherein the core of the particles has a density of at least 2500 kg/m³. This reference also discloses a method for manufacturing a bisphenol compound, comprising reacting a phenol and a ketone in the presence such a catalyst and wherein the reaction is performed in an up-flow reactor. The present inventors found that the mechanical stability of the core-shell ion exchange resin catalyst in presence of the reaction mixture at reaction temperature might be compromised under pressure or fluidization in the reactor. In particular, the inventors considered that the bond between the shell and the core is relevant to the long-term stability of the catalyst.

### SUMMARY

Accordingly, it is an object of the present invention to provide an improved ion exchange resin catalyst that is mechanically stable along with long term adhesion stability of the catalytic layer to the core material,

It is a further object of the present invention to provide for an ion exchange resin catalyst that can provide an improved selectivity towards a desired bisphenol compound, in particular an improved selectivity towards formation of p,p-bisphenol A.

Yet a further object of the invention is to provide an ion-exchange resin catalyst that can be used at weight hourly space velocities well above 1.0.

Yet a further object of the present invention is to provide an ion-exchange resin catalyst that can be used either in a fixed bed up-flow or in a fixed bed down-flow reactor.

The present inventors have found a method wherein core-shell ion exchange resin catalyst particles can be manufactured such that the shell, comprised of the ion-exchange resin, is chemically bonded to the core particle.

Accordingly, the present invention relates to a method for the manufacture of a core-shell catalyst comprising the steps of
a. providing core particles,
b. functionalizing at least part of the surface of the core particles with a functionalizing agent thereby forming functionalized core particles,
c. graft polymerizing at least one of aromatic vinyl compounds onto the functionalized core particles thereby forming core-shell particles wherein the core is comprised of the core particles and the shell is comprised of graft polymerized aromatic vinyl compounds and
d. activating the shell,
   wherein the core particles comprises or consists of glass particles and wherein prior to step b, the core particles are hydroxylated in the presence of a base.
   By application of the invention, the foregoing objects are met, at least in part.

The invention will now be described in more detail.

### BRIEF DESCRIPTION OF THE DRAWINGS

The above and other advantages and features of this disclosure will become more apparent by describing in further detail exemplary embodiments thereof with reference to the accompanying drawings, in which:

FIG. 1 illustrates a possible reaction taking place during silylation with 3-(trimethoxysilyl) propyl methacrylate (MPS);

FIG. 2 illustrates a possible reaction taking place during graft polymerization of polystyrene onto functionalized core particles;

FIG. 3 illustrates a possible reaction taking place during sulfonation of a grafted polymerized styrene;

FIG. 4 are Scanning Electron Microscope (SEM) images of a grafted polymerized styrene samples up to third cycle of stability test of Example 1;

FIG. 5 shows thermal gravimetric analysis (TGA) data for Example 1 after complete removal of unbound polystyrene (PS) by Soxhlet extraction and after each cycle of adhesion stability test;

FIG. 6 shows EDS mapping of a cross-sectional image for polymerized styrene grafted onto a glass bead sample of Example 1;

FIG. 7 shows SEM-EDAX analysis of samples of Example 2 before and after sulfonation;

FIG. 8 shows a comparison of catalytic performance of the manufactured core-shell catalyst of Example 2 with commercially available benchmark catalyst;

FIG. 9 shows selectivity towards p,p-BPA formation with respect to o,p-BPA of Example 2; and

FIG. 10 shows a comparison of performance of a comparative example IER catalyst vis-à-vis a core shell catalyst for bisphenol A (BPA) conversion up to sixth cycle.

### DETAILED DESCRIPTION

### Core particles

The core particles of the core shell catalyst serve two main purposes. First, the use of core particles allow the increase of the overall density of the catalyst particle, which is especially desired, in the case for use of the core-shell catalyst in an upflow reactor wherein a fixed bed operation is desired. Secondly, the core particles provide a substrate onto which a relatively thin layer of ion exchange resin can be graft polymerized. The use of such a thin layer was found to contribute to an improved selectivity. Thus, compared to commercially available ion-exchange resin beads, such core shell particles allow for a higher selectivity towards the formation of p,p-bisphenol A upon reacting acetone and phenol in the presence of said catalyst.

The core particles comprises or consists of glass particles.

Glass in particular includes silicate glass such as natural or synthetic quartz glass, boro-silicate glass, soda-lime-silicate glass, lead glass and alumino-silicate glass. Preferably glass beads are used in the present invention.

In the context of the present invention, the core particles are inert towards the reactants, reaction products and by-products typically used for the manufacture bisphenols, such as acetone, phenol and bisphenol A in particular. The core particles are preferably substantially spherical in shape meaning that a ratio Dₘₐₓ/Dₘᵢₙ is in the range of from 0.85-1.15, preferably from 0.95-1.05, more preferably 0.98-1.02, wherein Dₘₐₓ is a maximum diameter measured on the (core) particle and Dₘᵢₙ a minimum diameter measured on the (core) particle. Generally Dₘₐₓ/Dₘᵢₙ will be from 0.99-1.01. Most preferably, Dₘₐₓ/Dₘᵢₙ is 1.00 meaning that the core particles are perfect spheres having monodispersed distribution.

The core particles have an average particle diameter of 200-2000 μm, preferably 500-1000 μm as determined with microscopy. The average particle diameter of the core particles can be determined by commonly known techniques such as by microscopy methods, laser diffraction and sieving techniques. A preferred microscopy method is SEM (Scanning Electron Microscopy). The skilled person is well aware of such techniques. For the avoidance of doubt, it is noted that the average particle diameter is to be understood as the number based average. The average particle diameter may be determined by measuring, using microscopy such as SEM, the individual particle diameter of 25 particles followed by calculating the numerical average.

In the context of the invention, the core particles have a density of 2000 kg/m³ or more, preferably 2500 kg/m³ or more, such as 3000 kg/m³ or more. Suitably, the core particles have a density of 10000 kg/m³ or less, preferably 9000 kg/m³ or less, such as 8000 kg/m³ or less. Preferably, the density of the core particles is from 2000-4000 kg/m³.

### Hydroxylation

In accordance with the invention, the core particles, preferably glass beads, are first hydroxylated prior to being functionalized. Hydroxylation increases the amount of hydroxyl groups on the surface of the glass which allows for a more effective functionalization which in turn results in an improved adherence of the shell to the core.

Hydroxylation can be effected by treating the surface of the particles with an acidic or a basic solution. A preferred basic solution comprises a strong base such as sodium-hydroxide, potassium-hydroxide or cesium-hydroxide. A preferred acidic solution comprises a strong acid such as for example sulphuric acid, nitric acid, hydrochloric acid or phosphoric acid. A particularly usable solution is known as piranha solution which is a solution comprising sulphuric acid and hydrogen peroxide in molar ratio's of from 3:1 to 7:1. Other aqueous solutions of inorganic or organic bases may be useful provided they result in an increased amount of hydroxyl groups on the surface of the core particles.

Hydroxylation is effected by contacting the surface of the glass with the acidic or basic solution as described above for a certain amount of time and preferably at an increased temperature, i.e. temperatures of from 40-90° C. The time of hydroxylation depends on the temperature as well as on the strength of the base or acid that is used.

Following the step of contacting the glass surface of the core particles with the hydroxylation solution the glass surface of the core particles is preferably dried, for example by first washing the core particles with water and thereafter by acetone or a C1-C4 alcohol. The present invention is not strictly limited to this however and as long as an amount of hydroxylation is maintained any method of drying (if any) may be applied.

In order to check whether hydroxylation has occurred the skilled person has at its disposal known analytical techniques. For example, the FT-IR based ‘Philips Lighting method’ can be applied, the details of which can be found in the publication “Bert Sloots, Measuring the low OH content in quartz glass, Vibrational Spectroscopy, Volume 48, Issue 1, 2008, Pages 158-161, ISSN 0924-2031”.

In addition to analytical techniques the present inventors observed that if no or insufficient hydroxylation took place that there was an insufficient amount of grafting of vinyl aromatic polymer onto the particles which could be visually observed in that the particles would remain substantially transparent. To the contrary, if there was sufficient hydroxylation then there would be sufficient grafting of polymer resulting in the glass core particles no longer being transparent.

For the avoidance of doubt it is noted that the present invention is directed in step a) to the provision of core particles which is then followed by hydroxylation of the same prior to said hydroxylated particles being functionalised. The present invention however is also directed to the provision in step a) of hydroxylated core particles, which implies that hydroxylation has taken place prior to the hydroxylated core particles being functionalised.

### Functionalization of the Core Particles

At least part of the surface of the core particles is functionalized with a functionalizing agent thereby forming functionalized core particles. The functionalizing agent preferably forms a covalent bond with the surface of the core particle substrate thereby forming a functionalized coating on the core particle. Other types of bonding, such as ionic bonding, van der Waals bonding or hydrogen-bridge bonding may be suitable, yet are less preferred.

### Functionalizing Agent

The functionalizing agent is preferably a silane, which typically have the ability to form a chemical bond between organic and inorganic materials. In the present invention, the silane-functionalizing agent is represented by the following general formula (1),

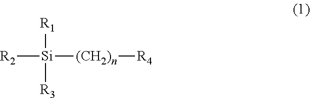

wherein,
R₁, R₂ and R₃ may be the same or different, and are independently selected from the group consisting of hydrogen, halogen, alkyl, haloalkyl, alkylene, alkynyl, aromatic groups, substituted aromatic groups, heteroaromatic groups, ester, ether, acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide provided that at least one of R₁, R₂ and R₃ is an acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide,
R₄ is a functional group for reacting with the aromatic vinyl compounds, and
n is between 0-20.

The functionalizing agent comprises at least one functional group for reacting with the core particle and at least one functional group for reacting with one or more of an aromatic vinyl compounds. The functional group for reacting with the core particle typically is a hydrolysable group that can undergo hydrolysis. Following hydrolysis, a reactive silanol group is formed, which can condense with other silanol groups, for example, those on the surface of siliceous fillers, to form siloxane linkages. The other functional group for reacting with one or more of an aromatic vinyl compounds is a non-hydrolysable organic radical that may possess a functionality that imparts desired characteristics.

The functional group for reacting with the core particles is selected from the group consisting of allyl, alkoxy, alkynoxy, aryloxy, acyloxy, acrylate, methacrylate, isocyanate, urethane, carbamate, epoxy, carboxylic acid, carboxylic acid anhydride, carboxylate, hydroxy, thiol, amine, aminoalkyl, arylamine and amide, and combinations of two or more of the foregoing. For the functionalizing agent according to structure (1), at least one of R₁, R₂ and R₃ is selected from the above functional groups for reacting with the core particles. In a preferred embodiment of the invention, at least one of R₁, R₂ and R₃ is selected from alkoxy, alkynoxy, aryloxy, acyloxy and combinations of two or more of the foregoing. In a particular preferred embodiment of the invention, R₁, R₂ and R₃ are selected from alkoxy, alkynoxy, aryloxy, acyloxy and combinations of two or more of the foregoing.

The functional group for reacting with one or more of the aromatic vinyl compounds is selected from the group consisting of organic groups comprising ethylenically unsaturated groups, acrylate groups, methacrylate groups, aldehyde groups, amine groups, azide groups, alkyl groups, alkenyl groups, alkinyl groups, aryl groups, aralkyl groups, cycloalkyl groups, cycloalkylene groups, hydroxyl groups, carboxyl groups, dipodal silane groups and combinations of two or more of the foregoing groups. For the functionalizing agent according to structure (1), R₄ is selected from at least one of the above functional groups for reacting with one or more of the aromatic vinyl compound. In a preferred embodiment of the invention, the R₄ is selected from organic groups comprising ethylenically unsaturated groups, acrylate groups, methacrylate groups, aldehyde groups and combinations of two or more of the foregoing.

For the functionalizing agent according to structure (1), n denotes the linker length between the organic functionality and the silicon atom. The linker length is an important factor for the physical property and reactivity of the silane-functionalizing agent. Preferably, the linker length, n=0-5, more preferably n=0-3 and most preferably n=3, a consequence of the fact that the propyl group is synthetically accessible, and has good thermal stability and facilitates chemical reactivity.

### Graft Polymerization

In accordance with the invention, after formation of the functionalized core particles, one or more aromatic vinyl compounds is graft polymerized onto the functionalized core particles, thereby forming core-shell particles wherein the core is comprised of said core particles and the shell is comprised of graft polymerized aromatic vinyl compounds.

The aromatic vinyl compound, in the present invention, comprises styrene and optionally a substituted styrene, preferably selected from the group consisting of alpha-methyl styrene, sulphonated styrene, vinyl toluene, ethyl vinyl benzene, vinyl naphthalene, or a combination thereof. Most preferably, the aromatic vinyl compound is a styrene.

The aromatic vinyl compound(s) react with the functional group R₄ of the silane-functionalizing agent according to structure (1), through a graft polymerization reaction. This result in the formation of covalent bonds between the in-situ polymerized aromatic vinyl compounds and the functionalizing agent. Through the process, the inventors have been able to manufacture graft polymerized aromatic vinyl compound(s), which, via the functionalizing agent is covalently bonded to the core particles. The layer comprised of the polymerized aromatic vinyl compound, together with the coating consisting of the functionalizing agent, forms the shell of the core shell particle.

In a preferred embodiment, the core particles comprises or consists of glass particles and the shell comprises or consists of graft-polymerized styrene. Styrene is graft polymerized onto functionalized glass particles, preferably through in-situ emulsion polymerization method. Emulsion polymerization method is well known to a person skilled in art. A suitable reactor to perform emulsion polymerization could be a continuous/batch stirred tank reactor or even a fluidized bed reactor. Alternatively, a bulk polymerization can be performed using hot nitrogen (or any other inert gas) to heat and fluidize the functionalized core particles. Functionalized core particles are initially charged into the reactor and fluidized with hot nitrogen preferably at 70 to 90° C. temperature. Then the styrene and the other monomer(s) can be introduced into the reactor at regular intervals to wet the functionalized core particles. As the particles are fluidized and heated with hot nitrogen, the adhered monomer mixture would polymerize to form a shell layer over the functionalized core particles. This process is repeated until the desired shell thickness is achieved over core particles.

Functionalized core particles are initially charged into the reactor and fluidized with hot nitrogen preferably at 70 to 90° C. temperature. Later, the styrene and the other monomer(s) can be introduced into the reactor at regular intervals to wet the functionalized core particles. As the particles are fluidized and heated with hot nitrogen, the adhered monomer mixture would polymerize to form a shell layer over the functionalized core particles. This process is repeated until the desired shell thickness is achieved over core particles.

A procedure to make core-shell particles is described by Gu et al. (Journal of Colloid and Interface Science 2004, 272 (2), 314-320). Although the original article applies this dispersion polymerization based method to prepare nano-sized core-shell particles (800-1500 nm), the present inventors have found that the method may also be applied to prepare micron sized core-shell catalyst particles after surface activation of the micron sized particles (200-2000 μm).

Additionally, the graft polymerization is preferably carried out in the presence of one or more of a cross-linking monomer. The crosslinking monomers include aromatic crosslinking monomers such as divinylbenzene, divinyltoluene, trivinylbenzene, divinyl chlorobenzene, diallyl phthalate, divinylnaphthalene, divinyl xylene, divinylethylbenzene, trivinyl naphthalene and polyvinylanthracenes; and aliphatic crosslinking monomers such as di-and polyacrylates and methacrylates exemplified by trimethylolpropane trimethacrylate, ethylene glycol dimethacrylate, ethylene glycol diacrylate, neopentyl glycol dimethacrylate and pentaerythritol tetra-and trimethacrylates, and trivinylcyclohexane. The crosslinking monomer is preferably present at levels from about 0.1 to about 20 weight percent of the total monomer, and more preferably from about 1 to about 10 weight percent of the total monomer. Preferred crosslinking monomers are; aromatic crosslinking monomers, and particularly preferred is divinylbenzene. In the obtained polymer shell, the degree of cross-linking is preferably from 1-4%, more preferably less than 2% degree of crosslinking.

### Activation

After graft polymerization of the aromatic vinyl compound(s) onto the functionalized core particles, the resulting core-shell particles are activated. Activation of the shell provides the core-shell particles with catalytic activity. The activation comprises sulfonating at least part of the graft polymerized aromatic vinyl compound using a sulfonating agent. The introduction of sulfonic acid groups not only yields catalytically active functional groups but also accomplishes sulfone crosslinking of the polymer in which the aromatic ring contains more than one sulfonic acid group per ring. In a preferred embodiment, the catalyst comprises sulfonic acid functional groups.

Optionally, the shell can additionally comprise an attached promoter, preferably a mercaptan compound, which is attached to catalyst by reacting with sulfonic acid functional groups. A mercaptan compound refers to a compound having a free form of SH group in the molecule. As the mercaptan, an alkyl mercaptan can be used (which may be a non-substituted alkyl mercaptan or an alkyl mercaptan having at least one substituting group, such as a carboxylic group, an amino group, a hydroxyl group, etc.). Examples of non-substituted alkyl mercaptan include methyl mercaptan, ethyl mercaptan, n-butyl mercaptan, and n-octyl mercaptan. Examples of substituted alkyl mercaptan include mercaptocarboxylic acids (such as thioglycolic acid and 3-mercaptopropionic acid), aminoalkane thiols (such as 2-amino ethane thiol and 2,2-dimethyl thiazolidine), and mercaptoalcohols (such as mercaptoethanol). Among these, the non-substituted alkyl mercaptans are preferred in terms of the promoting action. These mercaptans may be used singly or in combination. The promoter is attached to the resin forming the shell of the core-shell catalyst. Presence of an attached promotor allows the manufacture of bisphenol compounds without the need for addition of a separate promoter. Such technology is preferred as it reduced the presence of sulfur containing species in the final product such as in particular bisphenol A.

In accordance with the invention, the shell of the core-shell particles need not necessarily cover the core completely. For example, the shell may cover 50% or more of the surface area of the core, such as 60% or more, 70% or more, 80% or more, 90% or more, or 95% or more. Preferably, the core of the core-shell particles is completely covered with shell material.

Preferably, the shell of the core-shell catalyst has an average catalyst layer thickness 10-500 μm, preferably from 100-300 μm. The average layer thickness of the shell of the core-shell particles can be determined by, e.g., commonly known microscopy methods, automated imaging, laser diffraction and sieving techniques. A preferred method is SEM (Scanning Electron Microscopy). For the avoidance of doubt, it is noted that several ways of determining the shell thickness exist and that a skilled person will have no difficulty in accurately determining the average shell thickness with the average being a numerical average. A convenient method involves first measuring the average diameter of the core particles prior to functionalization. Then after graft polymerisation the average particle diameter is measured again and the average shell thickness can be calculated on the basis of the difference between the average diameter of the core particles and the average diameter of the core-shell particles.

### Manufacture of a Bisphenol Compound Using Core-Shell Catalyst

In a further aspect, the invention is directed to a method for the manufacture of a bisphenol compound comprising reacting a phenol and a ketone in the presence of the core-shell catalyst as prepared by the above-mentioned steps. In the said method, the reaction is performed in an up-flow reactor, wherein, the reactants are fed to the reactor at an inlet and bisphenol compound is extracted at an outlet at a position higher than the inlet, or in a down-flow reactor, wherein the reactants are fed to the reactor at an inlet and bisphenol compound is extracted at an outlet at a position lower than the inlet.

The bisphenol compound may, for instance, be bisphenol A (2,2′-bis(4-hydroxyphenyl)propane), bisphenol S (4,4′-sulphonyldiphenol), or bisphenol F (4,4′-dihydroxydiphenylmethane). Preferably, the bisphenol compound is bisphenol A. The molar ratio of phenol to acetone is usually in the range of 3-30 mol of phenol per mol of acetone, and preferably 5-15 mol of phenol per mol of acetone. If the molar ratio is smaller than 3 mol of phenol per mol of acetone, then the reaction speed is likely to be too slow. If it molar ratio is larger than 30 mol of phenol per mol of acetone, then the system becomes too dilute to have commercial significance.

Typically, the reaction temperature may be 40-150° C., preferably 60-110° C., more preferably 50-80° C. The reaction may be performed batch-wise or continuously. Preferably, the reaction is performed in a fixed bed continuous reactor in which phenol and ketone (such as acetone) are continuously fed into a reactor filled with the catalyst of the invention to react them. The reactor may be a single reactor, or maybe two or more reactors that are connected in series. Optionally the reaction mixture is subjected to a step for removing the reaction by-product viz. water, with an objective of promoting the reaction kinetics using a suitable separation technique. Such optional separation maybe performed under reduced pressure using a distillation column. In general, such distillation is carried out at a pressure of 6.5-80 kPa and at a temperature of 70-180° C. Unreacted phenol is then removed as an azeotrope. The bisphenol product may be concentrated by further removal of phenol. Such further distillation may typically be performed at 100-170° C. and a pressure of 5-70 kPa.

Optionally, the in-situ dehydration can also be achieved using a BPA stripping reactor using hot nitrogen or any other inert gas to strip water out of the reactor to promote reaction kinetics.

Optionally, the final product stream of the reactors may be further subjected to a separation step to remove unreacted acetone, water & a part of phenol to concentrate the BPA product stream.

In yet another aspect, the invention is directed to the use of the catalyst in a catalyst bed of an up-flow or a down-flow reactor for the manufacture of a bisphenol compound. Preferably the catalyst bed is a fixed bed. The catalyst of the invention increases bed stability, minimizes back mixing, and allows to operate the reactor at higher space velocity. Furthermore, the invention is directed towards the use of the catalyst for the manufacture of bisphenol A by reacting phenol with acetone for increasing the selectivity towards formation of p,p-bisphenol A.

The present invention will now be elucidated by means of the following non-limiting example(s) in detail.

### Materials Used and Their Sources

Glass beads were supplied by Retsch (product code 22.222.0004), 3-(trimethoxysilyl) propyl methacrylate (MPS), ethanol, sodium hydroxide (NaOH), styrene, sodium styrene sulfonate (NaSS), potassium persulfate (KPS) and divinyl benzene (DVB) were purchased from Sigma-Aldrich (India) and were used as received.

### Preparation of the Core-Shell Catalyst

### Step 1: Hydroxylation of Glass Surface—Core Particles

Transparent glass beads (20 g) were added to a two-necked 100 mL round bottom (RB) flask equipped with an overhead stirrer and condenser and 25 mL of 5M NaOH was added to it. The mixture was stirred using an overhead stirrer for the next 24 hours at 90° C. The reaction mixture was cooled to room temperature and subsequently, quenched by adding excess deionized (DI) water. The glass beads were removed and washed with DI water until the pH of the aqueous phase became neutral. Finally, the glass beads were washed with acetone and dried under vacuum at room temperature.

### Step 2: Silylation With MPS—Functionalized Core Particles

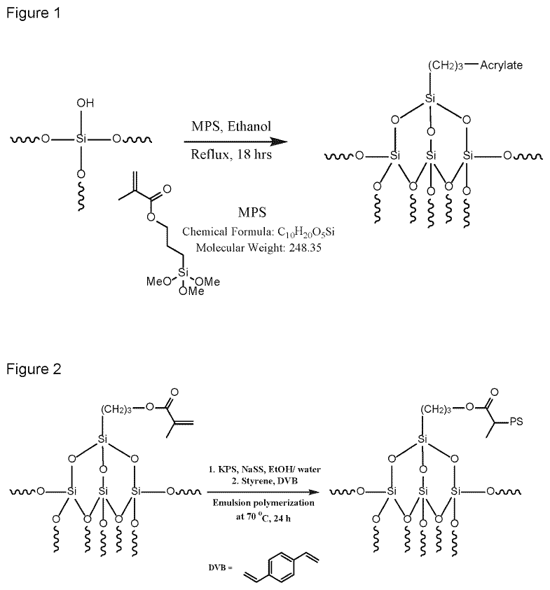

Cleaned and dried glass bead samples (6.0 g) as obtained at the end of step 1 were added to a two-necked 100 mL round bottom flask, equipped with an overhead stirrer and a condenser. MPS (7.1 mL) followed by 20 mL of ethanol was added to the flask. The mixture was stirred under reflux for 18 hours and then the resulting silylate-functionalized glass beads were washed several times (3×50 mL, for 30 minutes with stirring) with ethanol to completely remove any excess or unreacted MPS adhered to the glass bead surface. Finally, the silylate-functionalized glass beads were dried under vacuum at room temperature. The present inventors believe the reaction taking place during the functionalization of this Step 2 is as shown in FIG. 1.

### Step 3: Graft Polymerizing Polystyrene Onto the Functionalized Core Particles—Shell

In a 100 mL three necked round bottom flask, KPS (0.27 g) and NaSS (27 mg) was dissolved in water (5.5 mL) and was charged with nitrogen using a purge tube for 15 minutes. Silylate-functionalized glass beads as obtained at the end of Step 2 (5 g) was charged into the RB flask under nitrogen atmosphere. Ethanol (10.2 mL) was added under nitrogen atmosphere and left for purging for another 15 minutes. The mixture was heated to 70° C. and then styrene (5.8 mL) was added to the reaction mixture under nitrogen atmosphere. After 30 minutes of stirring using overhead mechanical stirrer, DVB solution (115 uL) in 2 mL ethanol was added to the reaction mixture. The reaction mixture was then left at 70° C. under nitrogen atmosphere for 24 hours under continuous stirring. After that, the emulsion was removed by repetitive washing with water. The samples were stirred in chloroform (CHCl₃, 70 mL) for 18 hours to extract any free polystyrene (PS) that is not covalently bound to the glass bead surface. Finally, the obtained core-shell particles were washed with methanol to remove any moisture and then dried under vacuum at room temperature. At this stage, the glass bead surface was found to be white in color showing grafting of polystyrene on the glass bead surface. The present inventors believe the reaction taking place during the functionalization of this Step 3 is as shown in FIG. 2.

### Step 4: Sulfonation of the Grafted Polymerized Styrene—Activation

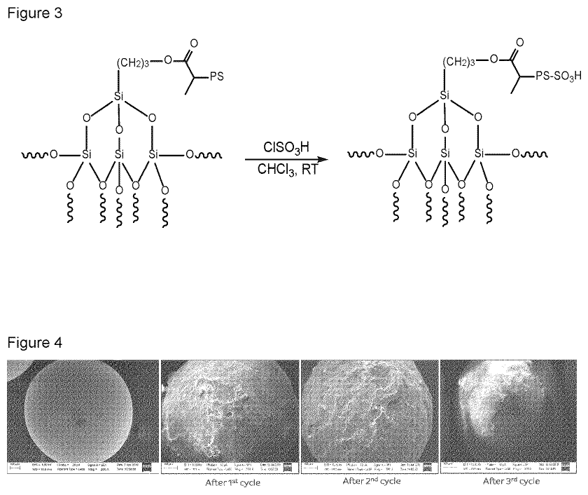

The core-shell particles obtained at the end of step 3 (1.0 g) were sulfonated by immersing the samples in in chloroform (15 mL) at room temperature for one hour under stirring using overhead mechanical stirrer followed by addition of an amount of chlorosulfonic acid (1.3 mole equivalent to the grafted PS content). The reaction mixture was then left for shaking for one hour in a mechanical shaker. After shaking for one hour, the resulting sulfonated core-shell particles were gradiently washed with chloroform (30 mL×3) to remove the excess acid present in the reaction mixture. After washing with chloroform, the sulfonated core-shell particles were further washed with mixture of chloroform and water (20 mL water×3). Finally, the sulfonated core-shell particles was washed with acetone and dried under vacuum at room temperature. The present inventors believe the reaction taking place during the functionalization of this Step 4 is as shown in FIG. 3.

### Comparative Example 1 (Without Hydroxylation)

In this example glass beads were silylated with MPS under the same condition as mentioned in Step 2 and Step 3 without however performing the hydroxylation of the glass surface as mentioned in Step 1. The particles obtained at the end of step 3 were examined visually to detect if polystyrene was grafted on the core particles. It was found that the glass beads remained transparent and that no, or at least negligible visible grafting of polystyrene on the surface glass bead surface had occured.

### Example 1

In this example, the experimental particles obtained at the end of Step 3 (hydroxylated and polymerized styrene grafted core-shell particles) were analyzed to determine the adhesion stability of the grafted polymerized styrene onto the glass beads. These particles were subjected to Soxhlet extraction using chloroform as solvent to remove any trace amount of unreacted styrene and other ingredients. Unbound PS particles were further removed from the dried mixture by using sieves. The obtained particles were used as test sample particles for adhesion stability test as follows.

Initial weight of the test particles were measured at the onset of the adhesion stability test (as-prepared weight). Then the adhesion stability of these test particles were examined for multiple cycles. One cycle of adhesion stability test represents stirring the test sample particles in the mixture of phenol and acetone (96:4, w/w) at 70° C. for 24 hours, washing with acetone for complete removal of phenol, drying under vacuum, and re-weighing the same. Dried samples after each cycle of adhesion stability test was also characterized by microscopy (SEM image analysis) and thermal gravimetric analysis (TGA) to monitor the adhesion stability of grafted polymerized styrene.

For the SEM analysis, the polymerized styrene grafted glass bead samples were gold coated to around 5 nm in thickness and then was observed in a ZEISS make ‘EVO18’ Scanning Electron Microscope (SEM). A comparison of the surface morphology of original glass beads and polymerized styrene grafted glass beads (collected after multiple cycles of adhesion stability test) allowed the inventors to analyze the uniformity of PS coating around the glass surface and adhesion stability of the same. SEM images of the grafted polymerized styrene samples up to third cycle of stability test is shown in FIG. 4. It was apparent from the images that the in-situ polymerized styrene remain grafted onto the glass bead surface after treated with mixture of phenol and acetone (96:4. w/w) at 70° C. for 24 hours. This demonstrates a high adhesion stability of the grafted polymerized styrene layer even after several cycles.

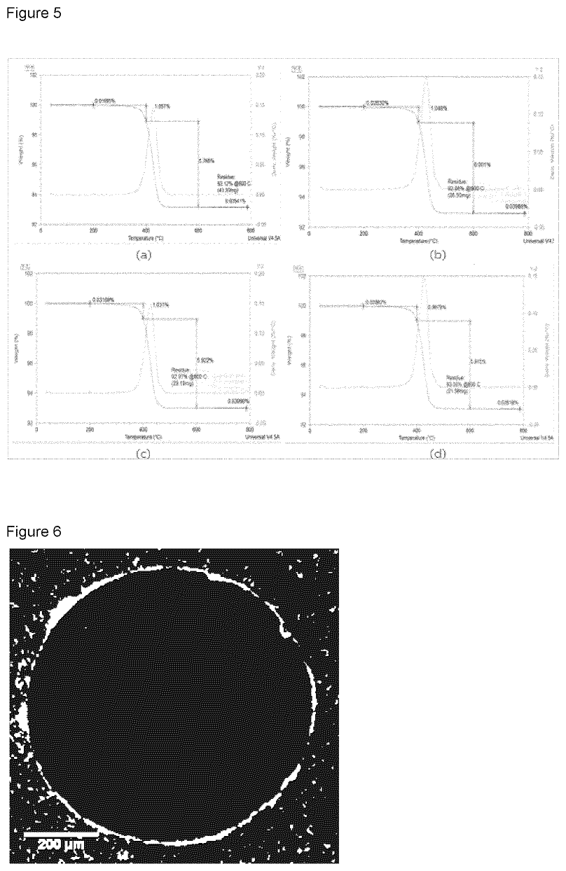

Further, the quantity of polymerized styrene grafted onto the glass bead surface was estimated by thermogravimetric analysis (TGA) by quantifying the weight loss due to grafted PS. The TGA of dried grafted polymerized styrene glass bead sample particles collected after each cycle of adhesion stability test were performed on a TA Instruments TGA Q5000 in air at a heating rate of 20° C. min 1 in the range of room temperature (RT) to 800° C. Weight loss due to complete degradation of organic content (grafted PS layer) in the range of 200° C. to 600° C. were determined for sample particles after each cycle of adhesion stability test to determine the adhesion stability of grafted PS content onto glass bead surface. The grafted polymerized styrene samples after recovery, washing and drying showed no loss in weight indicating highly stable adhesion of PS onto the glass bead surface. TGA data for the above-mentioned sample after complete removal of unbound PS by Soxhlet extraction and after each cycle of adhesion stability test has been shown in FIG. 5.

It is evident from the TGA analysis that polymerized styrene grafted onto the glass bead surface started degrading after 250° C. and complete combustion happened by 500° C. This further confirms the presence of polymerized styrene on the glass bead surface. Considering the possibility of inhomogeneity of grafted polystyrene around glass beads, average of the TGA data for six different samples was taken to monitor the quantity of polymerized styrene grafted onto the glass bead surface. The quantity of grafted polystyrene for all the samples analyses has been provided in Table 1. Thus, the TGA results also confirmed that the quantity of polymerized styrene grafted onto glass bead remained unchanged to a great extent, even after recovery, washing and drying of the samples from each cycle of adhesion stability test. This indicates excellent adhesion stability of the polymerized styrene grafted onto glass bead surface in the mixture of phenol and acetone (96:4, w/w) at 70° C. during and after multiple cycles.

TABLE 1
Quantity of polymerized styrene grafted onto the glass bead surface

| Sample* | Grafted PS content (wt. %) |
|---|---|
| As prepared | 7.5 ± 0.6 |
| After 1st cycle of stability test | 6.7 ± 0.8 |
| After 2nd cycle of stability test | 7.1 ± 0.8 |
| After 3rd cycle of stability test | 6.6 ± 0.4 |

EDS mapping of the SEM image also investigated the coverage of glass beads by the grafted polystyrene. EDS or EDAX (Energy-dispersive X-ray spectroscopy) analysis was performed on the gold coated samples using an Oxford make “X-Max-50 SDD” EDS system. The cross sectional image of the grafted PS around the glass bead particles was determined by EDS mapping using the same system. Polymerized styrene grafted glass bead particles were embedded in ‘Epo-thin’ epoxy using a “SimpliMet XPS1” automatic compression mounting system, 150° C. and 300 bar pressure was applied. Samples were then precisely polished to the middle of the glass beads using an “EcoMet 300” automated grinder polisher at room temperature. The embedded sample block was then polished using a silica paper (1000 grit) gently pressing against the cloth. Water was used to reduce the abrasion due to polishing. Both the sample block were then gold coated of about 2.5 nm in thickness and characterized using the said SEM and EDS system. ‘Image J’ was used for image processing.

EDS mapping of the cross-sectional image for the polymerized styrene grafted onto the glass bead sample has been shown in FIG. 6. In this figure, the dark color in the center represents glass bead core and the white outside border around the center represents grafted polystyrene moiety. It was apparent from the figure that around the spherical glass bead a thin layer of polystyrene has been coated covering almost all the glass surface. The thickness of the coating was largely non-uniform.

### Example 2

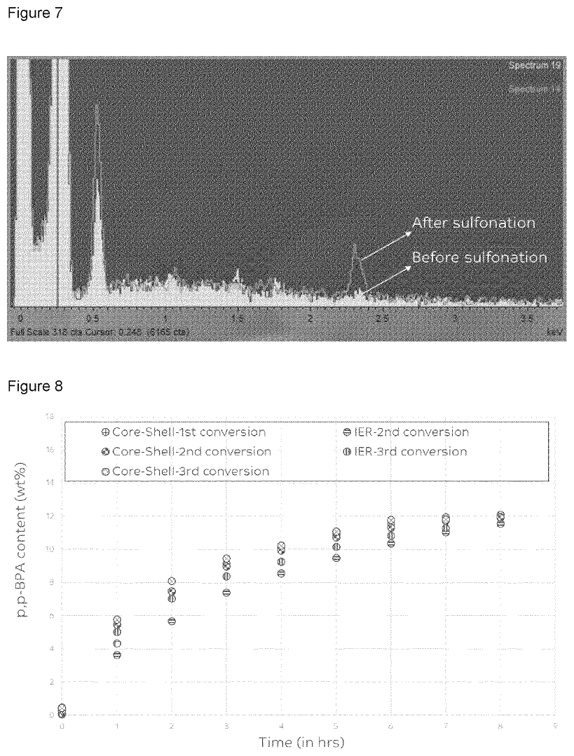

In this example, the samples as obtained at the end of Step 4 (sulfonated polymerized styrene grafted core-shell particles) were analyzed to confirm sulfonation. SEM-EDAX analysis of the samples before and after sulfonation is shown below in FIG. 7. In the figure, the two lines denote samples of polymerized styrene grafted onto the glass bead sample before sulfonation and after sulfonation, as denoted in the spectrum.

TABLE 2
Elemental analysis of polymerized styrene
grafted core-shell particles:

| Element | Before sulfonation (Wt. %) | After sulfonation (Wt. %) |
|---|---|---|
| C | 95.6 | 93.8 |
| O | 4.4 | 5.3 |
| S | 0.0 | 0.88 |

It was observed that after sulfonation reaction, the peak (at 2.3 unit) intensity corresponding to the signal of sulfur increased significantly compared to the sample before sulfonation. This confirmed the presence of sulfur indicating sulfonation of PS moiety. Sulfonation was further confirmed by elemental analysis (Table 2).

### Example 3

In this example, the catalyst performance of the core-shell ion exchange resin catalyst (as prepared following Steps 1-4) was evaluated. BPA conversion reaction from the mixture of phenol and acetone (96:4, w/w) using the manufactured core-shell type ion exchange resin catalyst particles (as obtained at the end of Step 4) was carried out at 70° C. for multiple times to evaluate the repeatability and reproducibility of the catalyst performance. Reactions were continued for 8 hours and samples were taken in an interval of one hour to understand the reaction kinetics. Commercially available LEWATIT K-1131® (IER) beads were used as a comparative example. The LEWATIT K-1131@ IER catalyst was pre-dried at 95° C. under vacuum for 8 hours and stored inside phenol for 12 hours to pre-swell prior to use.

In a typical reaction, 20 gm of reaction mixture contains 96 wt. % phenol and 4 wt. % acetone with the loading of 2 wt. % catalyst. In case of manufactured core-shell catalyst, the quantity of the shell was only considered as catalyst and same amount of IER (LEWATIT K-1131®) was used as comparative example. A comparison of catalytic performance of the manufactured core-shell catalyst with commercially available benchmark catalyst is shown in FIG. 8. It was observed that the catalytic activity of the newly developed catalyst demonstrated excellent catalytic performance in terms of consistent yield of p,p-BPA during multiple repetitive cycles. It also seemed to show a also higher conversion rate as shown in p,p-BPA yield during early hours and high adhesion stability of the covalently grafted catalytic layer onto the glass bead surface.

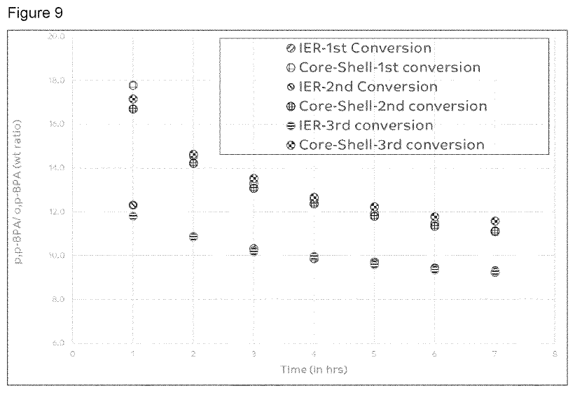

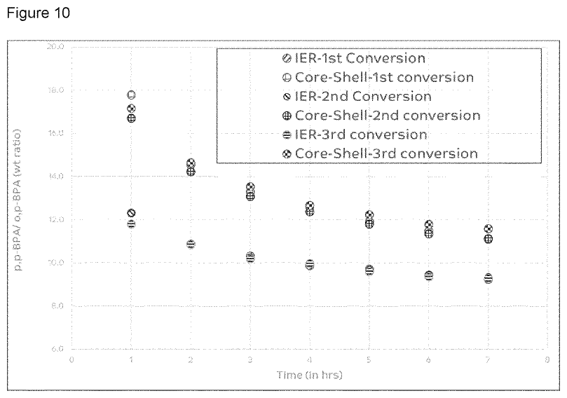

Selectivity towards p,p-BPA formation with respect to o,p-BPA is shown in FIG. 9. Advantage of a thin shell of catalytic layer around the solid core glass bead is again reflected in higher selectivity of p,p-BPA with respect to o,p-BPA and total mixture. The cycles were repeated thrice to confirm the repeatability of the catalytic performance. The performance of the comparative example IER catalyst vis-à-vis the core shell catalyst was compared for the BPA conversion up to sixth cycle, which is shown in FIG. 10. It was observed that the core shell catalyst according to the present invention showed the better performance in terms of yield and selectivity compared to IER based catalyst even up to sixth cycle. This observation demonstrated high integrity of the catalytic layer supported on glass beads.

1. Method for the manufacture of a core-shell catalyst comprising the steps of
a) providing core particles,
b) functionalizing at least part of the surface of the core particles with a functionalizing agent thereby forming functionalized core particles,
c) graft polymerizing one or more aromatic vinyl compounds onto the functionalized core particles thereby forming core-shell particles wherein the core is comprised of said core particles and the shell is comprised of graft polymerized aromatic vinyl compounds, and
d) activating the shell.
wherein the core particles comprise or consists of glass particles and wherein prior to step b, the core particles are hydroxylated.

2. The method of claim 1 wherein the core particles have an average particle diameter of 200-2000 μm, as determined with microscopy in accordance with the method described in the specification.

3. The method of claim 1, wherein the functionalizing agent comprises at least one functional group for reacting with the core particle and at least one functional group for reacting with said one or more aromatic vinyl compounds.

4. The method of claim 1, wherein
the functional group for reacting with the core particles is selected from the group consisting of allyl, alkoxy, alkynoxy, aryloxy, acyloxy, acrylate, methacrylate, isocyanate, urethane, carbamate, epoxy, carboxylic acid, carboxylic acid anhydride, carboxylate, hydroxy, thiol, amine, aminoalkyl, arylammo and amide, and combinations of two or more of the foregoing, and/or
the functional group for reacting with said one or more aromatic vinyl compounds is selected from the group consisting of organic groups comprising ethylenically unsaturated groups, acrylate groups, methacrylate groups, aldehyde groups, amine groups, azide groups, alkyl groups, alkenyl groups, alkinyl groups, aryl groups, aralkyl groups, cycloalkyl groups, cycloalkylene groups, hydroxyl groups, carboxyl groups, dipodal silane groups and combinations of two or more of the foregoing groups.

5. The method of claim 1, wherein the functionalizing agent is a silane represented by the following general formula (1), 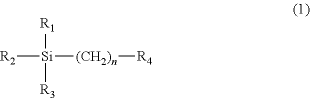
wherein
R₁, R₂ and R₃ may be the same or different, and are independently selected from the group consisting of hydrogen, halogen, alkyl, haloalkyl, alkylene, alkynyl, aromatic groups, substituted aromatic groups, heteroaromatic groups, ester, ether, allyl, alkoxy, alkynoxy, aryloxy, acyloxy, acrylate, methacrylate, isocyanate, urethane, carbamate, epoxy, carboxylic acid, carboxylic acid anhydride, carboxylate, hydroxy, thiol, amine, aminoalkyl, arylammo and amide provided that at least one of R₁, R₂ and R₃ is an acrylate, allyl, alkoxy, alkynoxy, aryloxy, acyloxy, methacrylate, isocyanate, urethane, carbamate, epoxy, carboxylic acid, carboxylic acid anhydride, carboxylate, hydroxy, thiol, amine, aminoalkyl, arylammo and amide,
R₄ is a functional group for reacting with the said aromatic vinyl compounds, and
n is between 0-20.

6. The method of claim 1, wherein the aromatic vinyl compound comprises styrene and optionally a substituted styrene.

7. The method of claim 1, wherein the graft polymerization is carried out in the presence of a cross-linking monomer.

8. The method of claim 1, wherein the activating step comprises sulfonating at least part of the graft polymerized aromatic vinyl compound using a sulfonating agent.

9. Catalyst obtained or obtainable by the method of claim 1.

10. The method of claim 1, wherein the shell has a thickness from 10-500 μm, as determined with microscopy in accordance with the method described in the specification.

11. Method for the manufacture of a bisphenol compound comprising reacting a phenol and a ketone in the presence of the catalyst of claim 9.

12. The method of claim 11 wherein the reaction is performed in a down-flow reactor or an up-flow reactor.

13. A method for the manufacture of a bisphenol compound comprising charging the catalyst of claim 9 into a catalyst bed of an up-flow or a down-flow reactor.

14. A method for the manufacture of bisphenol A comprising reacting phenol with acetone in the presence of the catalyst of claim 9 to increase selectivity towards formation of p,p-bisphenol A.
